(19) United States
(12) Patent Application Publication
(10) Pub. No.: US 2021/0169115 A1
(43) Pub. Date: Jun. 10, 2021

(54) NEONATAL PARENTERAL NUTRITION FORMULATIONS

(71) Applicant: LIVERPOOL WOMEN'S TRUST NHS FOUNDATION TRUST, Liverpool, Merseyside (GB)

(72) Inventors: Colin MORGAN, Liverpool, Merseyside (GB); Laura BURGESS, Liverpool, Merseyside (GB); Chandini PREMAKUMAR, Liverpool, Merseyside (GB); Shakeel HERWITKER, Liverpool, Merseyside (GB)

(21) Appl. No.: 17/052,361

(22) PCT Filed: May 1, 2019
(86) PCT No.: PCT/GB2019/051206

§ 371 Date: Nov. 2, 2020

(30) Foreign Application Priority Data
May 1, 2018 (GB) 1807155.5

(51) Int. Cl. A23L 33/175; A23L 33/00; A61K 31/195; A61K 47/18; A61K 9/00; A61P 3/02

(52) U.S. Cl. CPC A23L 33/175; A23L 33/40; A61P 3/02; A61K 47/183; A61K 9/0019; A61K 31/195

(57) ABSTRACT

The present invention relates to neonatal parenteral nutrition formulations. In particular, the present invention relates to neonatal parenteral nutrition formulations which comprise greater than 12% w/v arginine. Furthermore, the present invention also relates to the use of these neonatal parenteral nutrition formulations for use in the treatment of hypoargininaemia, hyperammonaemia, negative nitrogen balance or to prevent weight loss(i.e. to encourage weight gain) in neonates.

### INTRODUCTION

The present invention relates to neonatal parenteral nutrition formulations. In particular, the present invention relates to aqueous neonatal parenteral nutrition formulations, to methods and kits for making these formulations, to the use of these formulations for providing parenteral nutrition to neonates, especially pre-term and very pre-term neonates.

### BACKGROUND OF THE INVENTION

Human breast milk is the best and ideal form of nutrition for newborn babies, whether full-term or preterm neonates.¹ This is mainly because of the immense immunologic protection obtained from breast milk since immunologic defences of newborns are not fully developed.² Unfortunately, very preterm neonates (VPNs) are unable to digest nutritionally sufficient amounts of milk in their first few weeks of life and are totally or partially dependant on parenteral nutrition (PN).³⁻⁵

The aims of PN supply are to ensure provision of sufficient energy, not only to meet nutritional requirements, but also for growth and neurodevelopment.⁵ PN aims to supply nutrients at the same quantity and quality as the neonate would receive in utero (estimated as 15 g/kg/day³) or to match the needs of a foetus of the same post-conceptional age^{6,7} and also achieve satisfactory functional development. Increasing survival rates of ELBW premature neonates has made PN prescriptions more common and widely accepted.⁷ However, previous studies have indicated that current UK licensed neonatal parenteral nutrition amino acid formulations can routinely cause hypoargininaemia in PN dependant neonates.⁹ Thus, there remains a need for new PN formulations which are capable of mitigating the onset of hypoargininaemia in PN dependant neonates.

Arginine is one of many amino acids (AAs) present in a neonatal parenteral nutrition amino acid (AA) solution. Arginine is considered important in the VPN population due to its multiple roles in metabolic and inflammatory pathways and being a major component in body proteins.^{9,10} Arginine is mainly endogenously synthesised from the intestine and preterm neonates are known to have immature gut function. This means that, there is a lack of production of arginine that makes it a conditionally essential AA, requiring exogenous supply. This deficiency is further aggravated with the increased demand of arginine in preterm infants due to the multiple uses of arginine via various pathways such as for growth, ammonia detoxification, insulin secretion, precursors for synthesis of nitric oxide, creatine and polyamines which affect cardiovascular, pulmonary, immunological, intestinal and neurological function.

Recent evidence suggests that PN induced low plasma arginine levels are associated with hyperammonaemia¹¹ and, interestingly, studies supplementing preterm infants with exogenous intravenous L-arginine indicate that hyperammonaemia can be prevented and preterm morbidities, such as necrotising enterocolitis and persistent pulmonary hypertension, reduced¹⁰⁻¹⁴.

However, despite the above, neonatal parenteral AA formulations widely used in the UK, and elsewhere, have not been changed in the last 25 years to address these issues.⁹ Presently, only a very limited number of neonatal parenteral AA formulations exist with “high” arginine contents. An example of such a formulation would be the US licensed AA formulation known as TrophAmine 10% (Braun). TrophAmine 10% has an arginine content of 12% w/v, which is higher than many other commercial formulations such as Vaminolact 6.5% (Fresenius Kabi) and Primene 10% (Baxter) that have arginine contents of 6.3% and 8.4% respectively.^{9, 10} However, increasing the arginine content in AA formulations can result in undesired increases in nitrogen content, chloride content and instability to the formulation. Furthermore, AA formulations such as TrophAmine are still not freely available and the arginine concentrations they contain are still considered to be too “low” to suitably address conditions such as hypoargininaemia and hyperammonaemia in neonates.

Thus, there remains a need for new PN formulations for eonates which address many of the aforementioned problems.

The present invention was devised with the forgoing in mind.

### SUMMARY OF THE INVENTION

According to a first aspect of the present invention, there is provided an aqueous neonatal parenteral nutrition formulation comprising greater than 12% (w/v) arginine. The aqueous neonatal parenteral nutrition formulation suitably comprises greater than 14% (w/v) arginine.

Suitably, the aqueous neonatal parenteral nutrition formulation comprises greater than 14.5% (w/v) arginine or greater than 15% (w/v) arginine.

According to a second aspect of the present invention, there is provided an aqueous neonatal parenteral nutrition formulation as defined herein for use in the treatment of hypoargininaemia, hyperammonaemia, negative nitrogen balance or to prevent eight loss (i.e. to encourage weight gain) in neonates.

According to a third aspect of the present invention, there is provided an aqueous neonatal parenteral nutrition formulation as defined herein for use in reducing the incidence of infections and/or necrotising enterocolitis (NEC) in a neonate. Suitably, the neonate is a preterm or very preterm neonate.

According to a fourth aspect, the present invention provides a method of preparing an aqueous neonatal parenteral nutrition formulation as defined herein, the method comprising dissolving sufficient arginine in the aqueous medium to provide an aqueous neonatal parenteral nutrition formulation comprising greater than 12% (w/v) arginine.

Suitably, the method of the fourth aspect provides an aqueous neonatal parenteral nutrition formulation comprising greater than 14% (w/v) arginine

According to a fifth aspect, the present invention provides a method of preparing an aqueous neonatal parenteral nutrition formulation as defined herein, the method comprising adding a supplemental aqueous arginine solution comprising 10 to 50% (w/v), or 13 to 50% (w/v), or 15 to 50% (w/v), or 15 to 25% (w/v), arginine to a neonatal parenteral nutrition formulation comprising less than 12% (w/v) arginine to provide a final formulation comprising greater than 12% (w/v) arginine.

Suitably, the method of the fifth aspect comprises adding a supplemental aqueous arginine solution comprising 15 to 50% (w/v), or 15 to 25% (w/v), arginine to a neonatal parenteral nutrition formulation comprising less than 14% (w/v) arginine to provide a final formulation comprising greater than 14% (w/v) arginine

According to a sixth aspect of the present invention, there is provided a kit of parts comprising:
a) a 10-50% w/v, or 13 to 50% (w/v), or 15 to 50% (w/v), or 15 to 25% (w/v), aqueous solution of arginine; and
b) a neonatal parenteral nutrition formulation comprising less than 12% (w/v) arginine.

Suitably, the kit of parts comprises a neonatal parenteral nutrition formulation comprising less than 14% (w/v) arginine.

Suitably, the kit further comprises instructions on how to mix the aqueous solution of arginine with the neonatal parenteral nutrition formulation comprising less than 12% (w/v) arginine (or less than 14% arginine) to provide a neonatal parenteral nutrition formulation of the present invention.

Features, including optional, suitable, and preferred features in relation to one aspect of the invention may also be features, including optional, suitable and preferred features in relation to any other aspects of the invention.

### DETAILED DESCRIPTION OF THE INVENTION

### Definitions

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “parenteral” used herein will be understood to refer to the administration into the body (i.e. into the body of a neonatal) in a manner other than through the digestive tract. Typically, the term “parenteral” refers to administration to a human body via injection or infusion.

The term “neonate” used herein will be understood to refer to a newborn baby, specifically, to a newborn baby in the first four weeks after birth. The term “pre-term neonate” will be understood as referring to a baby born before 37 weeks gestational age.

In the context of the present invention, a “preterm” infant is understood to mean an infant born before 37 completed weeks of gestation (e.g. from 32 to 37 weeks gestation). In some embodiments the preterm infant may be born before 34 completed weeks of gestation.

In the context of the present invention, a “very preterm” infant is understood to mean an infant born with 28 to 32 completed weeks of gestation.

Where the quantity or concentration of a particular component is specified as a weight (or mass) percentage (% w/v), said weight (or mass) percentage refers to the percentage of said component by weight (or mass) relative to the total volume of the composition as a whole. It will be understood by those skilled in the art that the sum of weight (or mass) percentages of all components of a composition will total 100 wt %. However, where not all components are listed (e.g. where compositions are said to “comprise” one or more particular components), the weight (or mass) percentage balance may optionally be made up to 100 wt % by unspecified ingredients (e.g. a diluent, such as water, or other non-essentially but suitable additives).

### Formulations of the Present Invention

The inventors have developed an improved neonatal parenteral nutrition formulation which comprises increased concentrations of arginine and possesses good stability. Furthermore, the quantities of each of the other essential nutrients contained within formulation can be adjusted to ensure that the chloride and/or nitrogen content does not exceed acceptable/safe levels, while maintaining each of the essential nutrients at a concentration suitable and appropriate for neonatal administration.

The inventors have shown that the formulations of the present invention can greatly reduce the risk of hypoargininaemia. The inventors also surprisingly discovered that the formulations of the present invention are associated with enhancement of metabolic pathways involved in the immune response, particularly in the first 10 days of life, thus potentially reducing the incidence of infections and/or necrotising enterocolitis in neonates.

Thus, as indicated above, the present invention provides an aqueous neonatal parenteral nutrition formulation comprising greater than 12% (w/v) arginine.

In an embodiment, the neonatal parenteral nutrition formulation comprises greater than 13% (w/v) arginine. Suitably, the neonatal parenteral nutrition formulation comprises greater than 14% (w/v) arginine. Most suitably, the neonatal parenteral nutrition formulation comprises greater than 14.5% (w/v) arginine.

In another embodiment, the neonatal parenteral nutrition formulation comprises between 12% (w/v) and 30% (w/v) arginine. Suitably, the neonatal parenteral nutrition formulation comprises between 12% (w/v) and 25% (w/v) arginine. More suitably, the neonatal parenteral nutrition formulation comprises between 12% (w/v) and 20% (w/v) arginine. Yet more suitably, the neonatal parenteral nutrition formulation comprises between 14% (w/v) and 20% (w/v) arginine. Even more suitably, the neonatal parenteral nutrition formulation comprises between 14% (w/v) and 18% (w/v) arginine. Still more suitably, the neonatal parenteral nutrition formulation comprises between 14% (w/v) and 16% (w/v) arginine. Most suitably, the neonatal parenteral nutrition formulation comprises 15% (w/v) arginine.

In another embodiment, the neonatal parenteral nutrition formulation comprises between 14% (w/v) and 30% (w/v) arginine. Suitably, the neonatal parenteral nutrition formulation comprises between 14% (w/v) and 25% (w/v) arginine. More suitably, the neonatal parenteral nutrition formulation comprises between 14% (w/v) and 20% (w/v) arginine. Yet more suitably, the neonatal parenteral nutrition formulation comprises between 14% (w/v) and 20% (w/v) arginine. Even more suitably, the neonatal parenteral nutrition formulation comprises between 14% (w/v) and 18% (w/v) arginine. Still more suitably, the neonatal parenteral nutrition formulation comprises between 14% (w/v) and 16% (w/v) arginine.

In another embodiment, the neonatal parenteral nutrition formulation comprises between 14.5% (w/v) and 30% (w/v) arginine. Suitably, the neonatal parenteral nutrition formulation comprises between 14.5% (w/v) and 25% (w/v) arginine. More suitably, the neonatal parenteral nutrition formulation comprises between 14.5% (w/v) and 20% (w/v) arginine. Yet more suitably, the neonatal parenteral nutrition formulation comprises between 14.5% (w/v) and 20% (w/v) arginine. Even more suitably, the neonatal parenteral nutrition formulation comprises between 14.5% (w/v) and 18% (w/v) arginine. Still more suitably, the neonatal parenteral nutrition formulation comprises between 14.5% (w/v) and 16% (w/v) arginine.

Together with the above noted amounts of arginine, the neonatal parenteral nutrition formulations of the present invention may also include one or more other amino acids. Any suitable amino acid that is considered safe and of nutritional benefit to a neonate may be present in the formulations of the present invention.

In an embodiment, the neonatal parenteral nutrition formulation comprises one or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine and/or taurine.

In another embodiment, the neonatal parenteral nutrition formulation comprises two or more, suitably three or more, more suitably four or more, even more suitably five or more and most suitably six or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine and/or taurine.

In a specific embodiment, the neonatal parenteral nutrition formulation comprises each of the following amino acids isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine and taurine.

In certain embodiments, the neonatal parenteral nutrition formulation comprises isoleucine, leucine, lysine, methionine, phenylalanine, threonine, tryptophan, valine, histidine, alanine, glycine, aspartic acid, cysteine, glutamic acid, proline, serine and tyrosine and optionally taurine, asparagine, acetylcysteine and ornithine.

In yet another embodiment, the neonatal parenteral nutrition formulation comprises each of the following amino acids in the amounts shown below:

| | | |
|---|---|---|
| 2.5 to 7.5% | (w/v) | isoleucine |
| 7.0 to 11.0% | (w/v) | leucine |
| 6.0 to 12.0% | (w/v) | lysine |
| 1.0 to 4.0% | (w/v) | methionine |
| 2.0 to 5.0% | (w/v) | phenylalanine |
| 2.5 to 7.0% | (w/v) | threonine |
| 0.5 to 3.5% | (w/v) | tryptophan |
| 3.5 to 8.0% | (w/v) | valine |
| 1.0 to 5.0% | (w/v) | histidine |
| 6.0 to 10.0% | (w/v) | alanine |
| 1.0 to 5.0% | (w/v) | glycine |
| 0 to 2.0% | (w/v) | asparagine |
| 3.5 to 7.0% | (w/v) | aspartic acid |
| 0 to 2.0% | (w/v) | acetylcysteine |
| 0 to 2.5% | (w/v) | cysteine |
| 7.0 to 12.0% | (w/v) | glutamic acid |
| 0 to 4.0% | (w/v) | ornithine |
| 2.5 to 10.0% | (w/v) | proline |
| 2.5 to 6.5% | (w/v) | serine |
| 0 to 1.5% | (w/v) | tyrosine |
| 0 to 1.5% | (w/v) | taurine |
| 12 to 20% | (w/v) | arginine. |

In a further embodiment, the neonatal parenteral nutrition formulation comprises each of the following amino acids in the amounts shown below:

| | | |
|---|---|---|
| 3.5 to 6.5% | (w/v) | isoleucine |
| 9.0 to 10.5% | (w/v) | leucine |
| 7.0 to 11.0% | (w/v) | lysine |
| 1.0 to 3.0% | (w/v) | methionine |
| 3.0 to 4.5% | (w/v) | phenylalanine |
| 3.0 to 6.0% | (w/v) | threonine |
| 1.0 to 2.5% | (w/v) | tryptophan |
| 5.0 to 7.5% | (w/v) | valine |
| 2.0 to 4.0% | (w/v) | histidine |
| 6.5 to 9.5% | (w/v) | alanine |
| 2.5 to 4.0% | (w/v) | glycine |
| 0 to 1.0% | (w/v) | asparagine |
| 4.5 to 6.0% | (w/v) | aspartic acid |
| 0 to 1.0% | (w/v) | acetylcysteine |
| 0.5 to 2.0% | (w/v) | cysteine |
| 8.5 to 10.5% | (w/v) | glutamic acid |
| 0 to 3.5% | (w/v) | ornithine |
| 2.5 to 8.0% | (w/v) | proline |
| 3.0 to 6.0% | (w/v) | serine |
| 0 to 1.0% | (w/v) | tyrosine |
| 0 to 1.0% | (w/v) | taurine |
| 14 to 16% | (w/v) | arginine. |

In still another embodiment, the neonatal parenteral nutrition formulation comprises each of the following amino acids in the amounts shown below:

| | | |
|---|---|---|
| 2.5 to 7.5 | g/L | isoleucine |
| 6.5 to 11.0 | g/L | leucine |
| 5.0 to 12.0 | g/L | lysine |
| 1.0 to 6.0 | g/L | methionine |
| 2.0 to 6.0 | g/L | phenylalanine |
| 3.0 to 6.0 | g/L | threonine |
| 1.0 to 3.0 | g/L | tryptophan |
| 3.0 to 9.0 | g/L | valine |
| 1.5 to 4.5 | g/L | histidine |
| 5.5 to 12.0 | g/L | alanine |
| 1.5 to 16.0 | g/L | glycine |
| 0 to 2.0 | g/L | asparagine |
| 3.5 to 8.0 | g/L | aspartic acid |
| 0 to 2.5 | g/L | acetylcysteine |
| 0 to 2.5 | g/L | cysteine |
| 6.0 to 12.0 | g/L | glutamic acid |
| 0 to 4.0 | g/L | ornithine |
| 4.0 to 6.0 | g/L | proline |
| 1.0 to 5.0 | g/L | serine |
| 0 to 1.0 | g/L | tyrosine |
| 0 to 4.0 | g/L | taurine. |

Suitably, each amino acid in the neonatal parenteral nutrition formulation of the present invention is an L-amino acid.

Neonates also require certain essential minerals and/or electrolytes from their parenteral food source. Accordingly, in certain embodiments, the neonatal parenteral nutrition formulations of the present invention may also comprise one or more electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate.

In an embodiment, the neonatal parenteral nutrition formulations of the present invention comprise two or more, three or more, four or more, five or more, or six or more electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate.

In a particular embodiment, the neonatal parenteral nutrition formulations of the present invention comprise each of the following electrolytes: sodium, potassium, magnesium, calcium, selenium, zinc, copper, manganese, phosphate, sulphate, chloride, fluoride, iodide, acetate, citric acid, malate, lactate, glycerophosphate and gluconate.

In certain embodiments, the neonatal parenteral nutrition formulation comprises sodium, potassium, magnesium, calcium, sulphate, chloride, acetate, glycerophosphate and gluconate, and optionally, selenium, zinc, copper, manganese, phosphate, fluoride, iodide, citric acid, malate and lactate.

In further embodiments, the neonatal parenteral nutrition formulation comprises sodium, potassium, magnesium, calcium, sulphate, chloride, acetate, glycerophosphate, gluconate, selenium, zinc, copper, manganese, fluoride and iodide, and optionally, phosphate, citric acid, malate and lactate.

In a particular embodiment, the neonatal parenteral nutrition formulation comprises each of the following electrolytes in the amounts shown below:

| | | |
|---|---|---|
| 0.5 to 160 | mmol/L | sodium |
| 0.5 to 80 | mmol/L | potassium |
| 0.5 to 10 | mmol/L | magnesium |
| 0.5 to 120 | mmol/L | acetate |
| 0.5 to 120 | mmol/L | chloride |
| 0 to 10 | mmol/L | calcium |
| 0 to 20 | mmol/L | phosphate |
| 0 to 50 | mmol/L | glycerophosphate |
| 0 to 40 | mmol/L | selenium |
| 0 to 10 | mmol/L | zinc |
| 0 to 5 | mmol/L | copper |
| 0 to 50 | mmol/L | manganese |
| 0 to 50 | mmol/L | fluoride |
| 0 to 50 | mmol/L | iodide |
| 0 to 120 | mmol/L | gluconate |
| 0 to 50 | mmol/L | sulphate. |

In another particular embodiment, the neonatal parenteral nutrition formulation comprises each of the following electrolytes in the amounts shown below:

| | | |
|---|---|---|
| 0.5 to 160 | mmol/L | sodium |
| 0.5 to 80 | mmol/L | potassium |
| 0.5 to 10 | mmol/L | magnesium |
| 0.5 to 120 | mmol/L | acetate |
| 0.5 to 120 | mmol/L | chloride |
| 0.1 to 10 | mmol/L | calcium |
| 0.1 to 40 | mmol/L | selenium |
| 0.1 to 10 | mmol/L | zinc |
| 0.1 to 5 | mmol/L | copper |
| 0.1 to 50 | mmol/L | manganese |
| 0.1 to 20 | mmol/L | phosphate |
| 0.1 to 50 | mmol/L | fluoride |
| 0.1 to 50 | mmol/L | iodide |
| 0.1 to 50 | mmol/L | glycerophosphate |
| 0.1 to 120 | mmol/L | gluconate |
| 0.1 to 50 | mmol/L | sulphate. |

In yet another particular embodiment, the neonatal parenteral nutrition formulation comprises each of the following electrolytes in the amounts shown below:

| | | |
|---|---|---|
| 0.5 to 100 | mmol/L | sodium |
| 0.5 to 50 | mmol/L | potassium |
| 0.5 to 10 | mmol/L | magnesium |
| 0.5 to 100 | mmol/L | acetate |
| 0.5 to 100 | mmol/L | chloride |
| 0.5 to 10 | mmol/L | calcium |
| 0.5 to 30 | mmol/L | selenium |
| 0.5 to 5 | mmol/L | zinc |
| 0.1 to 2 | mmol/L | copper |
| 0.5 to 20 | mmol/L | manganese |
| 0.5 to 15 | mmol/L | phosphate |
| 0.5 to 15 | mmol/L | fluoride |
| 0.5 to 20 | mmol/L | iodide |
| 0.5 to 40 | mmol/L | glycerophosphate |
| 0.5 to 100 | mmol/L | gluconate |
| 0.5 to 40 | mmol/L | sulphate. |

In a particular embodiment, the neonatal parenteral nutrition formulation comprises each of the following electrolytes in the amounts shown below:

| | | |
|---|---|---|
| 60 to 160 | mmol/L | sodium |
| 10 to 40 | mmol/L | potassium |
| 1.5 to 5 | mmol/L | magnesium |
| 30 to 80 | mmol/L | acetate |
| 10 to 50 | mmol/L | chloride |
| 10 to 20 | mmol/L | calcium |
| 20 to 50 | mmol/L | phosphate |
| 20 to 50 | mmol/L | glycerophosphate |
| 0 to 2 | mmol/L | selenium |
| 0 to 1 | mmol/L | zinc |
| 0 to 0.5 | mmol/L | copper |
| 0 to 1 | mmol/L | manganese |
| 0 to 0.5 | mmol/L | fluoride |
| 0 to 0.5 | mmol/L | iodide |
| 0 to 100 | mmol/L | gluconate |
| 0 to 50 | mmol/L | sulphate. |

In a particular embodiment, the neonatal parenteral nutrition formulation comprises each of the following electrolytes in the amounts shown below:

| | | |
|---|---|---|
| 60 to 160 | mmol/L | sodium |
| 10 to 30 | mmol/L | potassium |
| 1.5 to 3 | mmol/L | magnesium |
| 30 to 50 | mmol/L | acetate |
| 10 to 80 | mmol/L | chloride |
| 10 to 30 | mmol/L | calcium |
| 20 to 30 | mmol/L | phosphate |
| 20 to 30 | mmol/L | glycerophosphate |
| 0.1 to 1 | mmol/L | selenium |
| 0.01 to 0.1 | mmol/L | zinc |
| 0.01 to 0.1 | mmol/L | copper |
| 0.05 to 0.5 | mmol/L | manganese |
| 0.01 to 0.1 | mmol/L | fluoride |
| 0 to 0.1 | mmol/L | iodide |
| 0 to 75 | mmol/L | gluconate |
| 0 to 30 | mmol/L | sulphate. |

In a further embodiment of the present invention, the neonatal parenteral nutrition formulation comprises one or more carbohydrates. Suitably, the neonatal parenteral nutrition formulation comprises one carbohydrate.

Any suitable carbohydrate suitable for neonatal nutrition may be used. A non-limiting list of possible carbohydrates include glucose, fructose, lactose and/or maltose. Suitably, the carbohydrate is glucose.

In an embodiment, the neonatal parenteral nutrition formulation comprises between 1 and 70% w/v of carbohydrate (e.g. glucose). Suitably, the neonatal parenteral nutrition formulation comprises between 1 and 50% w/v of carbohydrate (e.g. glucose). More suitably, the neonatal parenteral nutrition formulation comprises between 5 and 30% w/v of carbohydrate (e.g. glucose). Yet more suitably, the neonatal parenteral nutrition formulation comprises between 5 and 20% w/v of carbohydrate (e.g. glucose). Still more suitably, the neonatal parenteral nutrition formulation comprises between 10 and 20% w/v of carbohydrate (e.g. glucose). Most suitably, the neonatal parenteral nutrition formulation comprises between 10 and 15% w/v of carbohydrate (e.g. glucose).

It will be appreciated that together with the components listed above, the neonatal parenteral nutrition formulation of the present invention may include one or more additional ingredients. A non-limiting list of other ingredients which may be included in the neonatal parenteral nutrition formulations of the present invention include fats and fatty acids, salts, antibiotics, proteins, minerals and/or vitamins.

Suitable fats and fatty acids include long-chain polyunsaturated fatty acids (LCPUFAs) such as α-linoleic acid, eicosapentaenoic acid (EPA), arachidonic acid (ARA) and docosahexaenoic acid (DHA).

It is widely accepted that too much chloride in neonatal formulations can lead to hyperchloraemia and other related conditions. Thus, in a particular embodiment, the neonatal parenteral nutrition formulation of the present invention comprises less than 120 mmol/L of chloride. Suitably, the neonatal parenteral nutrition formulation of the present invention comprises less than 100 mmol/L of chloride. More suitably, the neonatal parenteral nutrition formulation of the present invention comprises less than 80 mmol/L of chloride. Yet more suitably, the neonatal parenteral nutrition formulation of the present invention comprises less than 50 mmol/L of chloride. Most suitably, the neonatal parenteral nutrition formulation of the present invention comprises less than 40 mmol/L of chloride.

In a further embodiment, the neonatal parenteral nutrition formulations of the present invention comprise between 4 g/L and 10 g/L of nitrogen. Suitably, the neonatal parenteral nutrition formulations of the present invention comprise between 4 g/L and 10 g/L of nitrogen. More suitably, the neonatal parenteral nutrition formulations of the present invention comprise between 4 g/L and 8 g/L of nitrogen. Even more suitably, the neonatal parenteral nutrition formulations of the present invention comprise between 5 g/L and 8 g/L of nitrogen. Most suitably, the neonatal parenteral nutrition formulations of the present invention comprise between 6 g/L and 7 g/L of nitrogen.

### Particular Embodiments

Particular embodiments of the present invention include:

1.1 a neonatal parenteral nutrition formulation comprising:
between 12% (w/v) and 20% (w/v) arginine;
three or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine and/or taurine;
three or more electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate; and
optionally one or more carbohydrates (e.g. glucose).

1.2 a neonatal parenteral nutrition formulation comprising:
between 14% (w/v) and 20% (w/v) arginine;
isoleucine, leucine, lysine, methionine, phenylalanine, threonine, tryptophan, valine, histidine, alanine, glycine, aspartic acid, cysteine, glutamic acid, proline, serine and tyrosine, and, optionally, taurine, asparagine, acetylcysteine and ornithine;
sodium, potassium, magnesium, calcium, sulphate, chloride, acetate, glycerophosphate and gluconate, and, optionally, selenium, zinc, copper, manganese, phosphate, fluoride, iodide, citric acid, malate and lactate; and
optionally one or more carbohydrates (e.g. glucose).

1.3 a neonatal parenteral nutrition formulation comprising:
between 14% (w/v) and 18% (w/v) arginine;
isoleucine, leucine, lysine, methionine, phenylalanine, threonine, tryptophan, valine, histidine, alanine, glycine, aspartic acid, cysteine, glutamic acid, proline, serine and tyrosine, and, optionally, taurine, asparagine, acetylcysteine and ornithine;
sodium, potassium, magnesium, calcium, sulphate, chloride, acetate, glycerophosphate and gluconate, and, optionally, selenium, zinc, copper, manganese, phosphate, fluoride, iodide, citric acid, malate and lactate; and
optionally one or more carbohydrates (e.g. glucose);
   wherein the neonatal parenteral nutrition formulation comprises less than 120 mmol/L of chloride and between 5 g/L and 8 g/L of nitrogen.

1.4 a neonatal parenteral nutrition formulation comprising:
each of the following amino acids in the amounts shown below:

| | | |
|---|---|---|
| 2.5 to 7.5% | (w/v) | isoleucine |
| 7.0 to 11.0% | (w/v) | leucine |
| 6.0 to 12.0% | (w/v) | lysine |
| 1.0 to 4.0% | (w/v) | methionine |
| 2.0 to 5.0% | (w/v) | phenylalanine |
| 2.5 to 7.0% | (w/v) | threonine |
| 0.5 to 3.5% | (w/v) | tryptophan |
| 3.5 to 8.0% | (w/v) | valine |
| 1.0 to 5.0% | (w/v) | histidine |
| 6.0 to 10.0% | (w/v) | alanine |
| 1.0 to 5.0% | (w/v) | glycine |
| 0 to 2.0% | (w/v) | asparagine |
| 3.5 to 7.0% | (w/v) | aspartic acid |
| 0 to 2.0% | (w/v) | acetylcysteine |
| 0 to 2.5% | (w/v) | cysteine |
| 7.0 to 12.0% | (w/v) | glutamic acid |
| 0 to 4.0% | (w/v) | ornithine |
| 2.5 to 10.0% | (w/v) | proline |
| 2.5 to 6.5% | (w/v) | serine |
| 0 to 1.5% | (w/v) | tyrosine |
| 0 to 1.5% | (w/v) | taurine |
| 14 to 20% | (w/v) | arginine; |

each of the following electrolytes in the amounts given:

| | | |
|---|---|---|
| 0.5 to 100 | mmol/L | sodium |
| 0.5 to 80 | mmol/L | potassium |
| 0.5 to 10 | mmol/L | magnesium |
| 0.5 to 120 | mmol/L | acetate |
| 0.5 to 120 | mmol/L | chloride |
| 0 to 10 | mmol/L | calcium |
| 0 to 40 | mmol/L | selenium |
| 0 to 10 | mmol/L | zinc |
| 0 to 5 | mmol/L | copper |
| 0 to 50 | mmol/L | manganese |
| 0 to 20 | mmol/L | phosphate |
| 0 to 50 | mmol/L | fluoride |
| 0 to 50 | mmol/L | iodide |
| 0 to 50 | mmol/L | glycerophosphate |
| 0 to 120 | mmol/L | gluconate |
| 0 to 50 | mmol/L | sulphate; and |

optionally one or more carbohydrates (e.g. glucose).

1.5 a neonatal parenteral nutrition formulation comprising:
each of the following amino acids in the amounts shown below:

| | | |
|---|---|---|
| 3.5 to 6.5% | (w/v) | isoleucine |
| 9.0 to 10.5% | (w/v) | leucine |
| 7.0 to 11.0% | (w/v) | lysine |
| 1.0 to 3.0% | (w/v) | methionine |
| 3.0 to 4.5% | (w/v) | phenylalanine |
| 3.0 to 6.0% | (w/v) | threonine |
| 1.0 to 2.5% | (w/v) | tryptophan |
| 5.0 to 7.5% | (w/v) | valine |
| 2.0 to 4.0% | (w/v) | histidine |
| 6.5 to 9.5% | (w/v) | alanine |
| 2.5 to 4.0% | (w/v) | glycine |
| 0 to 1.0% | (w/v) | asparagine |
| 4.5 to 6.0% | (w/v) | aspartic acid |
| 0 to 1.0% | (w/v) | acetylcysteine |
| 0.5 to 2.0% | (w/v) | cysteine |
| 8.5 to 10.5% | (w/v) | glutamic acid |
| 0 to 3.5% | (w/v) | ornithine |
| 2.5 to 8.0% | (w/v) | proline |
| 4.0 to 6.0% | (w/v) | serine |
| 0 to 1.0% | (w/v) | tyrosine |
| 0 to 1.0% | (w/v) | taurine |
| 14 to 16% | (w/v) | arginine; |

each of the following electrolytes in the amounts given:

| | | |
|---|---|---|
| 60 to 160 | mmol/L | sodium |
| 10 to 40 | mmol/L | potassium |
| 1.5 to 5 | mmol/L | magnesium |
| 30 to 80 | mmol/L | acetate |
| 10 to 50 | mmol/L | chloride |
| 10 to 20 | mmol/L | calcium |
| 20 to 50 | mmol/L | phosphate |
| 20 to 50 | mmol/L | glycerophosphate |
| 0 to 2 | mmol/L | selenium |
| 0 to 1 | mmol/L | zinc |
| 0 to 0.5 | mmol/L | copper |
| 0 to 1 | mmol/L | manganese |
| 0 to 0.5 | mmol/L | fluoride |
| 0 to 0.5 | mmol/L | iodide |
| 0 to 100 | mmol/L | gluconate |
| 0 to 50 | mmol/L | sulphate; |

between 5 and 20% w/v glucose.

In another particular embodiment of the present invention, the neonatal parenteral nutrition formulation has any one of the compositions provided under the headings “12% Arginine” and/or “15% Arginine” in Tables 1 and 2 hereinbelow.

Suitably, the neonatal parenteral nutrition formulation has any one of the compositions provided under the heading “15% Arginine” in Tables 1 and 2 hereinbelow.

### Applications

The formulations of the present invention are suitable for use in the treatment of any conditions associated with low levels of arginine (e.g. hypoargininaemia). Thus, the formulations are suitable for treating hypoargininaemia itself, as well as hyperammonaemia and a negative nitrogen balance. The formulations of the present invention can also be used to help prevent weight loss (i.e. to encourage weight gain) in neonates.

The formulations of the present invention have also been found to be useful for reducing the incidence of infections and/or NEC in neonates, see the examples hereinbelow.

Thus, according to one aspect of the present invention, there is provided an aqueous neonatal parenteral nutrition formulation as defined herein, for use in the parenteral nutrition for neonates.

According to another aspect of the present invention, there is provided an aqueous neonatal parenteral nutrition formulation as defined herein for use in the treatment of hypoargininaemia, hyperammonaemia, negative nitrogen balance or to prevent eight loss (i.e. to encourage weight gain) in neonates.

According to another aspect of the present invention, there is provided a method of treating hypoargininaemia, hyperammonaemia, negative nitrogen balance in neonates, or preventing weight loss (i.e. encouraging weight gain) in neonates, the method comprising administering an aqueous neonatal parenteral nutrition formulation as defined herein parenterally (e.g. intravenously) to the neonate.

According to another aspect of the present invention, there is provided an aqueous neonatal parenteral nutrition formulation as defined herein for use in reducing the incidence of infections and/or NEC in a neonate.

According to another aspect of the present invention, there is provided the use of an aqueous neonatal parenteral nutrition formulation as defined herein in combination with an intravenous solution comprising one or more lipids and/or vitamins in the treatment of hypoargininaemia, hyperammonaemia, negative nitrogen balance or to prevent weight loss (i.e. to encourage weight gain) in neonate. Suitably, the intravenous solution comprises one or more water-insoluble lipids and/or one or more water-insoluble vitamins.

According to another aspect of the present invention, there is provided a method of reducing the incidence of infections in a neonate, the method comprising administering an aqueous neonatal parenteral nutrition formulation as defined herein parenterally (e.g. intravenously) to the neonate.

Suitably, the formulations of the present invention are administered intravenously to the neonate. A clinician skilled in the field of neonatal medicine will be able to determine the appropriate rate and duration of administration for the formulations of the present invention, which will be analogous to the current regimes used for commercially available neonatal parenteral nutrition formulations.

Suitably, in the above applications, the neonate may be a pre-term neonate. Suitably, the neonate is a pre-term neonate born before 37 completed weeks of gestation or before 34 completed weeks of gestation. More suitably, the neonate is a pre-term neonate born before 34 completed weeks of gestation.

Suitably, in the above applications, the neonate may be a very pre-term neonate. Suitably, the very pre-term neonate is born before 32 completed weeks of gestation, before 30 completed weeks of gestation or before 29 completed weeks of gestation. Suitably, the neonate is a very pre-term neonate born before 30 completed weeks of gestation.

### Preparation of the Formulations of the Invention

The formulations of the present invention may be prepared by any suitable technique known in the art. For instance, the required amount of arginine may be dissolved within the aqueous medium of the formulation, along with other required/desired components to form a formulation of the present invention having greater than 12% (w/v) arginine.

Thus, according to one aspect of the present invention, there is provided a method of preparing an aqueous neonatal parenteral nutrition formulation as defined herein, the method comprising dissolving sufficient arginine in the aqueous medium to provide an aqueous neonatal parenteral nutrition formulation comprising greater than 12% (w/v) arginine.

It will be appreciated that other components identified above can also be dissolved in the aqueous medium to provide a resultant formulation having the desired amount/concentration of the component concerned.

Alternatively, a conventional neonatal formulation comprising less than 12% (w/v) arginine can be supplemented with arginine to provide a final formulation of the invention comprising greater than 12% (w/v) arginine.

Thus, according to another aspect, the present invention provides a method of preparing an aqueous neonatal parenteral nutrition formulation as defined herein, the method comprising adding a supplemental aqueous arginine solution comprising 10 to 50% (w/v), or 13 to 50% (w/v), or 15 to 50% (w/v), or 15 to 25% (w/v), to a neonatal parenteral nutrition formulation comprising less than 12% (w/v) arginine to provide a final formulation comprising greater than 12% (w/v) arginine.

According to a further aspect of the present invention, there is provided a kit of parts comprising:
a) a 10-50% w/v, or 13 to 50% (w/v), or 15 to 50% (w/v), or 15 to 25% (w/v), aqueous solution of arginine; and
b) a neonatal parenteral nutrition formulation comprising less than 12% (w/v) arginine.

Suitably the kit further comprises instructions on how to mix the aqueous solution of arginine with the neonatal parenteral nutrition formulation comprising less than 12% (w/v) arginine to provide a neonatal parenteral nutrition formulation of the present invention.

In an embodiment, the kit comprises a 10-40% w/v aqueous solution of arginine. Suitably, the kit comprises a 10-30% w/v aqueous solution of arginine. Suitably, the kit comprises a 13 to 50% (w/v) or 15 to 50% (w/v) aqueous solution of arginine. More suitably, the kit comprises a 15-25% w/v aqueous solution of arginine. Most suitably, the kit comprises a 20% w/v aqueous solution of arginine.

It will be understood that the neonatal parenteral nutrition formulation comprising less than 12% (w/v) arginine may be any preformed (e.g. commercially available) aqueous neonatal parenteral formulation. Suitable examples of such formulations include Vaminolact™ and/or Primene™. These commercial neonatal formulations are well-known in the art and their composition is given in the example section below.

In another embodiment, the kit may further comprise one or more additional parental nutrition products, such as, for example, Peditrace™.

### EXAMPLES

### Materials

### Product Formulation

See Tables 1 and 2 for formulations.

### Product Preparation

Products to be prepared by Pharmacy Aseptic Unit, Royal Liverpool & Broadgreen University Hospital.

### Testing

### Testing Scope

### Assay Development

Initial feasibility study will be performed by Biochemistry Department, Pathology Laboratories, Alder Hey Children's Hospital, to establish the capability of analysing the amino acid content of the formulations, using an Amino Acid Analyser.

### Stability Parameter

The shelf life will be determined by reviewing all data generated throughout the study for gross changes and against the needs and safety of the patient.

Due to the complex nature of the preparations and impact on accuracy and precision of analytical methods it may not be feasible to restrict shelf life to 5% degradation of critical Active Pharmaceutical Ingredients.

### Regulations and Guidelines

The study will be carried out following the guidelines laid out in the NHS yellow cover document (Standard Protocol for Deriving and Assessment of Stability, Part 4—Parenteral Nutrition, Edition 1, April 2016, NHS Pharmaceutical Quality Assurance Committee).

### Testing Methods

| | | | Time |
|---|---|---|---|
| | | Specifications/ | points |
| Test | Methods | Limits | tested |
| 1. Appearance | Visual | Against standard | Each time |
| | inspection | description | point |
| 2. pH | Of sample | Within 1.0 units of | Each time |
| | (SOP111) | initial time point. | point |
| 3. Optical | In-house method | Monitor for gross | Each time |
| Rotation | (SOP017) | changes in OR | point |
| 4. UV | In-house method | Monitor for gross | Each time |
| Absorbance | (SOP015) | changes in uv | point |
| | | absorbance. | |
| 5. Sub-visible | In-house method | Monitor for gross | Each time |
| particles | (SOP085) | changes and against | point |
| | | BP limits. | |
| 6. Amino Acid | In-house method | Monitor specific AA's | Each time |
| content | (SOP1107) | for gross change (<10% | point |
| | | change) | |

### Analytical Test Centre

Tests 1 to 5 to be performed at QCNW Liverpool Laboratory.

Test 6 to be performed at Biochemistry Laboratory, Pathology Department, Alder Hey Hospital.

### Method Development

Tests 1 to 5 will follow standard procedures. Standardised test methods will be established to monitor sample optical rotation and uv absorbance.

Amino acid analysis will be developed using blank sample preparations, Vaminolact and Primene. Blank samples will be prepared omitting Vaminolact and Primene.

### Method Validation

Validation of the test method for amino acids will be on those amino acids selected for analysis; based on review of formulations and test method.

### Sample Storage

Samples will be stored refrigerated and protected from light throughout the study.

### Sampling Protocol

At each time point each bag will be sampled and tested as shown in Section 6.2

### Quantity of Materials Required

### Method Validation

QCNW Liverpool:
1 bag of each preparation. 2.1.1; 2.1.3; 2.2.1 and 2.2.3

Biochemistry Department, Pathology Laboratories:
1 bag of each preparation. 2.1.1; 2.1.3; 2.2.1 and 2.2.3
1 blank bag of each preparation, omitting Vaminolact and Primene.
2 bottles of Vaminolact.
2 bottles of Primene.
2 bottles of Peditrace.

### Stability Study

Product 2.1.1: (Vaminolact 6.5% with Arginine 6.3%):

(Without Peditrace Addition)

1 bag for tests 1, 2, 3 and 4. (QCNW Liverpool)

1 bag for test 6. (Alder Hey)

(Without Peditrace Addition)

6 bags for test 5. (QCNW Liverpool)

Total number required=8 bags.

4.2: (With Peditrace addition)

1 bag for tests 1, 2, 3 and 4. (QCNW Liverpool)

1 bag for test 6. (Alder Hey)

4 bags for test 5. (QCNW Liverpool)

Total number required=6 bags.

Product 2.1.3: (Vaminolact 6.5% with Arginine 15%):

(Without Peditrace Addition)

1 bag for tests 1, 2, 3 and 4. (QCNW Liverpool)

1 bag for test 6. (Alder Hey)

(Without Peditrace Addition)

6 bags for test 5. (QCNW Liverpool)

Total number required=8 bags.

(With Peditrace Addition)

1 bag for tests 1, 2, 3 and 4. (QCNW Liverpool)

1 bag for test 6. (Alder Hey)

4 bags for test 5. (QCNW Liverpool)

Total number required=6 bags.

Product 2.2.1: (Primene 10% with Arginine 8.4%):

(Without Peditrace Addition)

1 bag for tests 1, 2, 3 and 4. (QCNW Liverpool)

1 bag for test 6. (Alder Hey)

(Without Peditrace Addition)

6 bags for test 5. (QCNW Liverpool)

Total number required=8 bags.

(With Peditrace Addition)

1 bag for tests 1, 2, 3 and 4. (QCNW Liverpool)

1 bag for test 6. (Alder Hey)

4 bags for test 5. (QCNW Liverpool)

Total number required=6 bags.

Product 2.2.1: (Primene 10% with Arginine 15%):

(Without Peditrace Addition)

1 bag for tests 1, 2, 3 and 4. (QCNW Liverpool)

1 bag for test 6. (Alder Hey)

(Without Peditrace Addition)

6 bags for test 5. (QCNW Liverpool)

Total number required=8 bags.

(With Peditrace Addition)

1 bag for tests 1, 2, 3 and 4. (QCNW Liverpool)

1 bag for test 6. (Alder Hey)

4 bags for test 5. (QCNW Liverpool)

Total number required=6 bags.

### Preparation of Study Samples

### Without Peditrac

These samples will be prepared at the start of the study time points (Time 0), with agreement between Test Sites and Preparation Site.

### With Peditrace

These samples will be prepared 4 weeks after the ‘Without Peditrace’ samples; the Peditrace will not be added at this stage. The samples will be held at the ‘Preparation Site’ for 84 days prior to addition of Peditrace and transport to ‘Test Sites’. Day zero test date will be on day of Peditrace addition.

Testing with Peditrace will be ‘worst case’, with Peditrace added 84 days after preparation of primary bags.

### Changes in the Composition of the Formulations

The PN formulation of the present invention was formulated using the current PN formulations as a basis. The existing practice at the Liverpool Women's Hospital is to use Vaminolact which contains 6.5% arginine to make the PN bags and also provide an amino acid intake of 2.3 g/kg/day gradually increased to a maximum of 3.8 g/kg/day.

As part of the study design, it was intended to increase arginine content without changing the overall nitrogen content of the PN formulation. To do this, it was essential to not just add on additional arginine to the existing solution because that would result in a drastic overall total nitrogen content increase but what had to be done was to reduce the volume of the Vaminolact solution in the bag and substitute that with additional arginine concentrate solution. For this, a 20% arginine hydrochloride solution was used. This solution has MHRA approval for use. The table below with the composition of the current formulation and new formulations well illustrate these changes in content.

Similarly, following these principles, the PN formulation using Primene instead of Vaminolact was designed. The Primene amino acid solution has an arginine content of 8.4% (g arginine per 100 g amino acid) which forms the control PN formulation whereas the intervention PN formulation has arginine content increased to 15%.

Once again, the same approach was used wherein the volume of Primene used was reduced and substituted by addition of arginine hydrochloride 20% solution and also water. The justification for formulating a PN solution with Primene was mainly to address the issue of high threonine levels in the Vaminolact solution. Currently, the neonates have extremely high threonine levels well above the reference range and since Primene has lower threonine content compared to Vaminolact, it will be worth to understand if this intervention proves to be useful for this population in the future. Hence, the stability testing schedule includes testing of this formulation.

The most significant changes between the control and intervention formulation include:
(i) Reduction in volume of Vaminolact and Primene used which is then balanced out by addition of arginine 20% and also water, to ensure final volume remains as 300 ml to ease administration and fluid rate settings.
(ii) Formulations with Primene as the base amino acid—volumes have once again changed to ensure overall nitrogen content remains the same.
(iii) Change from potassium chloride to potassium acetate to ensure chloride content is not too high since the new formulations with additional arginine will have extra chloride contributed from the arginine hydrochloride solution. This is important because biochemically the preterm neonates have been reported to develop hyperchloraemia even with the current PN formulation in the clinical setting.
(iv) The content of the amino acids change slightly in the 15% arginine content solutions compared to the control solutions due to the reduction in volume of the base amino acid solution (Vaminolact or Primene) used. This is a desirable effect indirectly as it reduced the content of essential amino acids which clinically have been shown to be above the reference range values for this population.

TABLE 1
Parenteral Nutrition Formulations Containing Vaminolact and Arginine Additions:

| VAMINOLACT 6.5% | | | 6.3% Arginine | 12% Arginine | 15% Arginine |
|---|---|---|---|---|---|
| Ingredient | | | Volume (mL) | Volume (mL) | Volume (mL) |
| Glucose 70% | | | 51 | 51 | 51 |
| Water for Injection | | | 21.8 | 27.9 | 31.8 |
| Vaminolact | | | 196 | 186 | 180 |
| Sodium Chloride 30% | | | 0 | 0 | 0 |
| Sodium Glycero Phosphate 21.6% | | | 6 | 6 | 6 |
| Sodium Acetate 30% | | | 0 | 0 | 0 |
| Potassium Acetate 49% | | | 1.2 | 1.2 | 1.2 |
| Magnesium Sulphate 10% | | | 1.6 | 1.6 | 1.6 |
| Calcium Gluconate 10% | | | 20 | 20 | 20 |
| Arginine 20% | | | 0 | 3.9 | 6 |
| Without Peditrace | | | 297.6 | 297.6 | 297.6 |
| Peditrace | | | 2.4 | 2.4 | 2.4 |
| Total Volume | | | 300 | 300 | 300 |
| | | | g/297.6 ml | g/297.6 ml | g/297.6 ml |
| Vaminolact | g/1000 ml | | product | product | product |
| Alanine Ph Eur | 6.3 | | 1.235 | 1.172 | 1.134 |
| Arginine Ph Eur 4.1 grams | 4.1 | | 0.804 | 1.543 | 1.938 |
| Aspartic acid Ph Eur 4.1 grams | 4.1 | | 0.804 | 0.763 | 0.738 |
| Cysteine/Cystine 1.0 grams | 1.0 | | 0.196 | 0.186 | 0.180 |
| Glutamic acid Ph Eur 7.1 grams | 7.1 | | 1.392 | 1.321 | 1.278 |
| Glycine BP 2.1 grams | 2.1 | | 0.412 | 0.391 | 0.378 |
| Histidine USP 2.1 grams | 2.1 | | 0.412 | 0.391 | 0.378 |
| Isoleucine Ph Eur 3.1 grams | 3.1 | | 0.608 | 0.577 | 0.558 |
| Leucine Ph Eur 7.0 grams | 7.0 | | 1.372 | 1.302 | 1.260 |
| Lysine 5.6 grams | 5.6 | | 1.098 | 1.042 | 1.008 |
| Methionine Ph Eur 1.3 grams | 1.3 | | 0.255 | 0.242 | 0.234 |
| Phenylalanine Ph Eur 2.7 grams | 2.7 | | 0.529 | 0.502 | 0.486 |
| Proline Ph Eur 5.6 grams | 5.6 | | 1.098 | 1.042 | 1.008 |
| Serine Ph Eur 3.8 grams | 3.8 | | 0.745 | 0.707 | 0.684 |
| Taurine 0.3 grams | 0.3 | | 0.059 | 0.056 | 0.054 |
| Threonine USP 3.6 grams | 3.6 | | 0.706 | 0.670 | 0.648 |
| Tryptophan USP 1.4 grams | 1.4 | | 0.274 | 0.260 | 0.252 |
| Tyrosine USP 0.5 grams | 0.5 | | 0.098 | 0.093 | 0.090 |
| Valine Ph Eur 3.6 grams | 3.6 | | 0.706 | 0.670 | 0.648 |
| | | | μg/300 ml of | μg/300 ml of | μg/300 ml of |
| Peditrace | μg/ml | μmol/ml | product | product | product |
| Zn | 250.00 | 3.82 | 600.0 | 600.0 | 600.0 |
| Cu | 20.00 | 0.315 | 48.0 | 48.0 | 48.0 |
| Mn | 1.00 | 18.20 | 2.40 | 2.40 | 2.40 |
| Se | 2.00 | 25.30 | 4.80 | 4.80 | 4.80 |
| F | 57.00 | 3.00 | 136.8 | 136.8 | 136.8 |
| I | 1.00 | 7.88 | 2.40 | 2.40 | 2.40 |
| | | | g/297.6 ml | g/297.6 ml | g/297.6 ml |
| | | | product | product | product |
| Nitrogen (g) | | | 1.82 | 1.94 | 2.00 |
| Protein (g) | | | 11.37 | 11.57 | 11.64 |
| Total kcalories from glucose | | | 142.8 | 142.8 | 142.8 |
| Total kcalories from Vaminolact and | | | 47.04 | 47.76 | 48 |
| Arg 20% | | | | | |
| Total kcalories | | | 189.84 | 190.56 | 190.8 |
| Total AA (g) | | | 12.80 | 12.93 | 12.95 |

TABLE 2
Parenteral Nutrition Formulations Containing Primene and Arginine Additions:

| PRIMENE 10% | | | 8.4% Arginine | 12% Arginine | 15% Arginine |
|---|---|---|---|---|---|
| Ingredient | | | Volume (mL) | Volume (mL) | Volume (mL) |
| Glucose 70% | | | 51 | 51 | 51 |
| Water for Injection | | | 102.8 | 104.5 | 105.6 |
| Primene | | | 115 | 111 | 108 |
| Sodium Chloride 30% | | | 0 | 0 | 0 |
| Sodium Glycerophosphate 21.6% | | | 6 | 6 | 6 |
| Sodium Acetate 30% | | | 0 | 0 | 0 |
| Potassium Acetate 49% | | | 1.2 | 1.2 | 1.2 |
| Magnesium Sulphate 10% | | | 1.6 | 1.6 | 1.6 |
| Calcium Gluconate 10% | | | 20 | 20 | 20 |
| Arginine 20% | | | 0 | 2.3 | 4.2 |
| Without Peditrace | | | 297.6 | 297.6 | 297.6 |
| Peditrace | | | 2.4 | 2.4 | 2.4 |
| Total Volume | | | 300 | 300 | 300 |
| | | | g/297.6 ml | g/297.6 ml | g/297.6 ml |
| Primene 10% | g/1000 ml | | product | product | product |
| L-Isoleucine | 6.70 | | 0.771 | 0.744 | 0.724 |
| L-Leucine | 10.00 | | 1.150 | 1.110 | 1.080 |
| L-Valine | 7.60 | | 0.874 | 0.844 | 0.821 |
| L-Lysine | 11.00 | | 1.265 | 1.221 | 1.188 |
| L-Methionine | 2.40 | | 0.276 | 0.266 | 0.259 |
| L-Phenylalanine | 4.20 | | 0.483 | 0.466 | 0.454 |
| L-Threonine | 3.70 | | 0.426 | 0.411 | 0.400 |
| L-Tryptophan | 2.00 | | 0.230 | 0.682 | 0.216 |
| L-Arginine | 8.40 | | 0.966 | 1.392 | 1.747 |
| L-Histidine | 3.80 | | 0.437 | 0.422 | 0.410 |
| L-Alanine | 8.00 | | 0.920 | 0.888 | 0.864 |
| L-Aspartic Acid | 6.00 | | 0.690 | 0.666 | 0.648 |
| L-Cysteine | 1.89 | | 0.217 | 0.210 | 0.204 |
| L-Glutamic Acid | 10.00 | | 1.150 | 1.110 | 1.080 |
| Glycine | 4.00 | | 0.460 | 0.444 | 0.432 |
| L-Proline | 3.00 | | 0.345 | 0.333 | 0.324 |
| L-Serine | 4.00 | | 0.460 | 0.444 | 0.432 |
| L-Tyrosine | 0.45 | | 0.052 | 0.050 | 0.049 |
| L-Ornithine Hydrochloride | 3.18 | | 0.366 | 0.353 | 0.343 |
| Taurine | 0.60 | | 0.069 | 0.067 | 0.065 |
| | | | μg/300 ml of | μg/300 ml of | μg/300 ml of |
| Peditrace | μg/ml | μmol/ml | product | product | product |
| Zn | 250.00 | 3.82 | 600.0 | 600.0 | 600.0 |
| Cu | 20.00 | 0.315 | 48.0 | 48.0 | 48.0 |
| Mn | 1.00 | 18.20 | 2.40 | 2.40 | 2.40 |
| Se | 2.00 | 25.30 | 4.80 | 4.80 | 4.80 |
| F | 57.00 | 3.00 | 136.8 | 136.8 | 136.8 |
| I | 1.00 | 7.88 | 2.40 | 2.40 | 2.40 |
| | | | g/297.6 ml | g/297.6 ml | g/297.6 ml |
| | | | product | product | product |
| Nitrogen (g) | | | 1.73 | 1.79 | 1.85 |
| Protein (g) | | | 11.39 | 11.45 | 11.53 |
| Total kcalories from glucose | | | 142.8 | 142.8 | 142.8 |
| Total kcalories from Primene and | | | 45.54 | 45.80 | 46.13 |
| Arg 20% | | | | | |
| Total kcalories | | | 188.34 | 188.60 | 188.93 |
| Total AA (g) | | | 11.5 | 11.56 | 11.64 |

Association of Low Plasma Arginine Levels with Infection in Very Preterm Infants Receiving Parenteral Nutrition

### Morgan C Burgess L

Objectives and study: It has previously been shown that very preterm infants (VPI) have low plasma arginine levels when receiving current parenteral nutrition (PN) amino acid (AA) formulations. Arginine is involved in several metabolic and inflammatory pathways including those affecting T-cell function. Hypoargininaemia is associated with necrotising enterocolitis (NEC). Arginine and glutamine share metabolic pathways. Glutamine also has a role in immune function and gut integrity.

Aim: To compare plasma arginine levels in VPIs stratified according to whether positive (PS) or negative for sepsis (NS).

Methods: The RCT: Standardised Concentrated Additional Macronutrient Parenteral (SCAMP) nutrition study⁽¹⁵⁾ stratified infants into to gestational bands and randomised to receive a standard or high protein/energy PN regimen. Our secondary analysis re-stratified VPIs into PS or NS based on blood culture in the first 28 days of life using previously published sepsis criteria and outcomes.⁽¹⁶⁾ This process was repeated for a positive/negative diagnosis of confirmed NEC and the composite outcome of NEC or sepsis. Plasma arginine, glutamine and glutamate levels were measured in the second week of life using ion exchange chromatography.

Results: Of the 150 VPI (<29 weeks) randomised in the original study, amino acid data was available for 47/57 in the PS group and 77/93 in the NS group. Mean (sd) plasma arginine levels were lower in the PS group (Table 3) and lowest in the 24-26 week gestation stratum. They were also lower in the substratum of all PS infants (n=21) with more than 1 episode of sepsis: 30 (15) μmol/l (p=0.02). Analysis was unchanged by excluding the 2 infants with early onset sepsis (day 1-3). There was no difference in plasma amino acids comparing NEC (n=19) and no NEC (n=105) groups. The composite outcome: NEC or sepsis is shown in Table 3.

TABLE 3
Mean (sd) plasma amino acid levels (μmol/l)

| Group (n) | Birthweight | PN age (d) | Arginine | Glutamine | Glutamate |
|---|---|---|---|---|---|
| PS: all infants (47) | 856 (148) | 9 (3) | 37 (18) | 442 (114) | 96 (32) |
| NS: all infants (77) | 915 (180) | 10 (3) | 48 (30) | 491 (161) | 112 (65) |
| p | 0.06 | 0.35 | 0.03 | 0.07 | 0.12 |
| PS: 24-26 wks (27) | 807 (114) | 9 (2) | 30 (11) | 439 (120) | 95 (29) |
| NS: 24-26 wks (32) | 798 (162) | 10 (3) | 46 (36) | 470 (167) | 121 (82) |
| p | 0.74 | 0.41 | 0.03 | 0.35 | 0.19 |
| PS or NEC (59) | 845 (157) | 9 (3) | 36 (19) | 447 (131) | 107 (46) |
| NS and no NEC (65) | 932 (173) | 10 (3) | 50 (31) | 493 (158) | 105 (63) |
| p | <0.01 | 0.25 | <0.01 | 0.08 | 0.88 |

Conclusion: Sepsis in the first 28 days of life shows a strong association with low plasma arginine levels in VPIs receiving PN.

PAINT Study: Arginine Intake and Plasma Arginine Levels (mmol/l): All Values Median (Range)

Plasma arginine levels are lower in infants with lower gestation/birthweight. Given these infants also spend longer on PN they are more likely to have persistent hypoargininaemia up to and beyond day 10 of life. The data below show 12% arginine is insufficient to correct day 10 hypoargininaemia in the majority of infants studied (even in a population that is lower risk than the control group). A PN formulation comprising 15% arginine avoided day 10 hypoargininaemia in 5/6 patients despite the group being relatively high risk of persistent hypoargininaemia. All groups demonstrate large variation in plasma arginine levels. Day 1 (D1) arginine supplementation did not appear to enhance day 3 (D3) or day 10 (D10) arginine levels (data not shown) compared to day 3 supplementation although numbers were small and variation high.

TABLE 4
Arginine intake and plasma arginine levels (mmol/l): all values median (range)

| | Control (6.2%) | | 12% Arginine | | 15% Arginine | |
|---|---|---|---|---|---|---|
| N | 8 | | 12 | | 6 | |
| N (supplemented day 1) | 0 | | 4 | | 2 | |
| Birthweight (g) | 840 | (665-1290) | 890 | (600-1230) | 705 | (610-910) |
| Gestation (weeks) | 26.4 | (23.8-28.8) | 26.8 | (24.4-28.7) | 25.0 | (23.1-30.1) |
| Plasma arginine levels | | | | | | |
| D 3 (excluding D 1 | 28 | (9-61) | 46 | (17-81) | 23 | (13-33) |
| supplmnt) | | | | | | |
| D 10 | 33 | (9-97) | 51 | (14-161) | 72 | (19-131) |
| Parenteral arginine | | | | | | |
| intake (D 8-10) | | | | | | |
| Actual % total AA | 6.2 | | 12.0 | (11.2-13.0) | 14.9 | (14.2-17.0) |
| Arginine (mg/kg/d) | 249 | (81-265) | 421 | (205-473) | 565 | (361-582) |
| Maximum parenteral | | | | | | |
| arginine intake | | | | | | |
| Arginine (mg/kg/d) | 268 | (236-271) | 467 | (437-500) | 592 | (560-645) |
| Day of life | 9 | (6-10) | 7 | (5-9) | 8.5 | (5-9) |

### PAINT Study: Microarray Analysis Preliminary Findings (12% Arginine PN Supplementation Only)

Microarray techniques allow the analysis of the expression of a large number of genes simultaneously. The gene expression profiles can then be compared to identify changes in gene expression between two groups or at different time-points. The gene expression profiles between arginine supplemented versus non-supplemented group would be compared and paired patient samples from day 3 and day 10 compared. The up- or down-regulation of certain genes can be used to highlight the pathways involved in the immune and metabolic consequences of hypoargininaemia.

Understanding the meaning of the differential gene expression requires complex bioinformatic statistical analysis. However, the preliminary data from 6% and 12% arginine PN formulations show changes in gene expression that affect key inflammatory pathways involved in the response to infection between day 3 and 10. These pathways affect the immune responses involving phagocytosis and B cell function. Effects on key inflammatory cytokines (interferon beta, IL-I and TGF beta) have also been identified. The effects are also closely related to plasma arginine level. Supplementation with arginine also has an effect, but this seems less than for plasma level or postnatal age. This is particularly important because we have already shown (see above) that the 15% Arginine PN supplementation is more successful in achieving satisfactory arginine levels in the very preterm population than current 12% arginine PN supplementation.

There is increasing interest in the role of factors that alter cell energy metabolism in the immune response and there is some evidence from changes in gene expression that arginine supplementation affects key enzymes in the TCA cycle and metabolism of pyruvate to acetyl-CoA.

While specific embodiments of the invention have been described herein for the purpose of reference and illustration, various modifications will be apparent to a person skilled in the art without departing from the scope of the invention as defined by the appended claims.

### REFERENCES

1. Lawrence P B. Breast milk. Best source of nutrition for term and preterm infants. Pediatric clinics of North America. 1994; 41(5):925-941.
2. Jackson K M, Nazar A M. Breastfeeding, the immune response, and long-term health. The Journal Of The American Osteopathic Association. 2006; 106(4):203-207.
3. Gargasz A. Neonatal and pediatric parenteral nutrition. AACN advanced critical care. 2012; 23(4):451-464; quiz 465-456.
4. Wardlaw T, Blanc A, Zupan J, Ahman E. Low Birthweight-Country, Regional and Global Estimates. World Health Organization; 2004.
5. Koletzko B, Goulet O, Hunt J, Krohn K, Shamir R. 1. Guidelines on Paediatric Parenteral Nutrition of the European Society of Paediatric Gastroenterology, Hepatology and Nutrition (ESPGHAN) and the European Society for Clinical Nutrition and Metabolism (ESPEN), Supported by the European Society of Paediatric Research (ESPR). Journal of pediatric gastroenterology and nutrition. 2005; 41 Suppl 2:S1-87.
6. Embleton N E, Pang N, Cooke R J. Postnatal Malnutrition and Growth Retardation: An Inevitable Consequence of Current Recommendations in Preterm Infants? Pediatrics. 2001; 107(2):270.
7. Hans D M, Pylipow M, Long J D, Thureen P J, Georgieff M K. Nutritional practices in the neonatal intensive care unit: analysis of a 2006 neonatal nutrition survey. Pediatrics. 2009; 123(1):51-57.
8. Agostoni C, Buonocore G, Carnielli V P, et al. Enteral nutrient supply for preterm infants: commentary from the European Society of Paediatric Gastroenterology, Hepatology and Nutrition Committee on Nutrition. Journal of pediatric gastroenterology and nutrition. 2010; 50(1):85-91.
9. Morgan C, Burgess L. High Protein Intake Does Not Prevent Low Plasma Levels of Conditionally Essential Amino Acids in Very Preterm Infants Receiving Parenteral Nutrition. JPEN J Parenter Enteral Nutr. 2015.
10. Morgan C. Early amino acid administration in very preterm infants: Too little, too late or too much, too soon? Semin Fetal Neonatal Med. 2013.
11. Wu G, Jaeger L A, Bazer F W, Rhoads J M. Arginine deficiency in preterm infants: biochemical mechanisms and nutritional implications. The Journal of nutritional biochemistry. 2004; 15(8):442-451.
12. Heird W C, Nicholson J F, Driscoll Jr J M, Schullinger J N, Winters R W. Hyperammonemia resulting from intravenous alimentation using a mixture of synthetic L-amino acids: A preliminary report. The Journal of Pediatrics. 1972; 81(1):162-165.
13. Amin H J, Zamora S A, McMillan D D, et al. Arginine supplementation prevents necrotizing enterocolitis in the premature infant. J Pediatr. 2002; 140(4):425-431.
14. McCaffrey M J, Bose C L, Reiter P D, Stiles A D. EFFECT OF L-ARGININE INFUSION ON INFANTS WITH PERSISTENT PULMONARY-HYPERTENSION OF THE NEWBORN. PEDIATRIC RESEARCH. 1995; 37(4):A341-A341.
15. Morgan C et al. Pediatrics 2014; 133:e120-8
16. Whitby T et al. Arch Dis Child Fetal Neonatal Ed 2015; 100:F250-2.

The following numbered clauses 1-25 are not claims, but instead serve to define particular aspects and embodiments of the invention:
1. A neonatal parenteral nutrition formulation comprising greater than 12% (w/v) arginine.
2. A neonatal parenteral nutrition formulation according to clause 1, wherein the formulation comprises between 12% (w/v) and 30% (w/v) arginine.
3. A neonatal parenteral nutrition formulation according to clauses 1 or 2, wherein the formulation comprises between 14% (w/v) and 20% (w/v) arginine.
4. A neonatal parenteral nutrition formulation according to any one of clauses 1 to 3, wherein the formulation comprises between 14% (w/v) and 16% (w/v) arginine.
5. A neonatal parenteral nutrition formulation according to any one of clauses 1 to 4, wherein the formulation comprises one or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine or taurine.
6. A neonatal parenteral nutrition formulation according to any one of clauses 1 to 5, wherein the formulation comprises three or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine or taurine.
7. A neonatal parenteral nutrition formulation according to any one of clauses 1 to 6, wherein the formulation comprises six or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine or taurine.
8. A neonatal parenteral nutrition formulation according to any one of clauses 1 to 7, wherein the formulation comprises each of the following amino acids in the amounts given:

| | | |
|---|---|---|
| 2.5 to 7.5% | (w/v) | isoleucine |
| 7.0 to 11.0% | (w/v) | leucine |
| 6.0 to 12.0% | (w/v) | lysine |
| 1.0 to 4.0% | (w/v) | methionine |
| 2.0 to 5.0% | (w/v) | phenylalanine |
| 2.5 to 7.0% | (w/v) | threonine |
| 0.5 to 3.5% | (w/v) | tryptophan |
| 3.5 to 8.0% | (w/v) | valine |
| 1.0 to 5.0% | (w/v) | histidine |
| 6.0 to 10.0% | (w/v) | alanine |
| 1.0 to 5.0% | (w/v) | glycine |
| 0 to 2.0% | (w/v) | asparagine |
| 3.5 to 7.0% | (w/v) | aspartic acid |
| 0 to 2.0% | (w/v) | acetylcysteine |
| 0 to 2.5% | (w/v) | cysteine |
| 7.0 to 12.0% | (w/v) | glutamic acid |
| 0 to 4.0% | (w/v) | ornithine |
| 2.5 to 10.0% | (w/v) | proline |
| 2.5 to 6.5% | (w/v) | serine |
| 0 to 1.5% | (w/v) | tyrosine |
| 0 to 1.5% | (w/v) | taurine |
| 12 to 18% | (w/v) | arginine |

9. A neonatal parenteral nutrition formulation according to any one of clauses 1 to 8, wherein the formulation comprises each of the following amino acids in the amounts given:

| | | |
|---|---|---|
| 3.5 to 6.5% | (w/v) | isoleucine |
| 9.0 to 10.5% | (w/v) | leucine |
| 7.0 to 11.0% | (w/v) | lysine |
| 1.0 to 3.0% | (w/v) | methionine |
| 3.0 to 4.5% | (w/v) | phenylalanine |
| 3.0 to 6.0% | (w/v) | threonine |
| 1.0 to 2.5% | (w/v) | tryptophan |
| 5.0 to 7.5% | (w/v) | valine |
| 2.0 to 4.0% | (w/v) | histidine |
| 6.5 to 9.5% | (w/v) | alanine |
| 2.5 to 4.0% | (w/v) | glycine |
| 0 to 1.0% | (w/v) | asparagine |
| 4.5 to 6.0% | (w/v) | aspartic acid |
| 0 to 1.0% | (w/v) | acetylcysteine |
| 0.5 to 2.0% | (w/v) | cysteine |
| 8.5 to 10.5% | (w/v) | glutamic acid |
| 0 to 3.5% | (w/v) | ornithine |
| 2.5 to 8.0% | (w/v) | proline |
| 3.0 to 6.0% | (w/v) | serine |
| 0 to 1.0% | (w/v) | tyrosine |
| 0 to 1.0% | (w/v) | taurine |
| 14 to 16% | (w/v) | arginine |

10. A neonatal parenteral nutrition formulation according to any one of clauses 1 to 9, wherein the formulation comprises one or more electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate.
11. A neonatal parenteral nutrition formulation according to any one of clauses 1 to 10, wherein the formulation comprises three or more, or preferably five or more, electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate.
12. A neonatal parenteral nutrition formulation according to any one of clauses 1 to 11, wherein the formulation comprises each of the following electrolytes in the amounts given:

| | | |
|---|---|---|
| 0.5 to 100 | mmol/L | sodium |
| 0.5 to 80 | mmol/L | potassium |
| 0.5 to 10 | mmol/L | magnesium |
| 0.5 to 120 | mmol/L | acetate |
| 0.5 to 120 | mmol/L | chloride |
| 0 to 10 | mmol/L | calcium |
| 0 to 40 | mmol/L | selenium |
| 0 to 10 | mmol/L | zinc |
| 0 to 5 | mmol/L | copper |
| 0 to 50 | mmol/L | manganese |
| 0 to 20 | mmol/L | phosphate |
| 0 to 50 | mmol/L | fluoride |
| 0 to 50 | mmol/L | iodide |
| 0 to 50 | mmol/L | glycerophosphate |
| 0 to 120 | mmol/L | gluconate |
| 0 to 50 | mmol/L | sulphate |

13. A neonatal parenteral nutrition formulation according to any one of clauses 1 to 12, wherein the formulation comprises each of the following electrolytes in the amounts given:

| | | |
|---|---|---|
| 0.5 to 100 | mmol/L | sodium |
| 0.5 to 80 | mmol/L | potassium |
| 0.5 to 10 | mmol/L | magnesium |
| 0.5 to 120 | mmol/L | acetate |
| 0.5 to 120 | mmol/L | chloride |
| 0.1 to 10 | mmol/L | calcium |
| 0.1 to 40 | mmol/L | selenium |
| 0.1 to 10 | mmol/L | zinc |
| 0.1 to 5 | mmol/L | copper |
| 0.1 to 50 | mmol/L | manganese |
| 0.1 to 20 | mmol/L | phosphate |
| 0.1 to 50 | mmol/L | fluoride |
| 0.1 to 50 | mmol/L | iodide |
| 0.1 to 50 | mmol/L | glycerophosphate |
| 0.1 to 120 | mmol/L | gluconate |
| 0.1 to 50 | mmol/L | sulphate |

14. A neonatal parenteral nutrition formulation according to any one of clauses 1 to 13, wherein the formulation comprises each of the following electrolytes in the amounts given:

| | | |
|---|---|---|
| 60 to 160 | mmol/L | sodium |
| 10 to 40 | mmol/L | potassium |
| 1.5 to 5 | mmol/L | magnesium |
| 30 to 80 | mmol/L | acetate |
| 10 to 50 | mmol/L | chloride |
| 10 to 20 | mmol/L | calcium |
| 20 to 50 | mmol/L | phosphate |
| 20 to 50 | mmol/L | glycerophosphate |
| 0 to 2 | mmol/L | selenium |
| 0 to 1 | mmol/L | zinc |
| 0 to 0.5 | mmol/L | copper |
| 0 to 1 | mmol/L | manganese |
| 0 to 0.5 | mmol/L | fluoride |
| 0 to 0.5 | mmol/L | iodide |
| 0 to 100 | mmol/L | gluconate |
| 0 to 50 | mmol/L | sulphate |

15. A neonatal parenteral nutrition formulation according to any one of clauses 1 to 14, wherein the formulation further comprises one or more carbohydrates.
16. A neonatal parenteral nutrition formulation according to clause 15 wherein the carbohydrate is glucose.
17. A neonatal parenteral nutrition formulation according to clause 16, wherein the formulation comprises between 5 and 20% w/v of glucose.
18. A neonatal parenteral nutrition formulation according to any preceding clause, characterised in that the formulation comprises less 120 mmol/L of chloride.
19. A neonatal parenteral nutrition formulation according to any preceding clause, wherein the formulation has any one of the compositions provided under the headings “12% Arginine” and/or “15% Arginine” in Tables 1 and 2.
20. A neonatal parenteral nutrition formulation according to any preceding clause, wherein the formulation has any one of the compositions provided under the heading “15% Arginine” in Tables 1 and 2.
21. An aqueous neonatal parenteral nutrition formulation according to any one of clauses 1 to 20, for use in the parenteral nutrition for neonates.
22. An aqueous neonatal parenteral nutrition formulation according to any one of clauses 1 to 20 for use in the treatment of hypoargininaemia, hyperammonaemia, negative nitrogen balance or to prevent weight loss (i.e. to encourage weight gain) in neonates.
23. A method of treating hypoargininaemia, hyperammonaemia, negative nitrogen balance in neonates, or preventing weight loss (i.e. encouraging weight gain) in neonates, the method comprising administering an aqueous neonatal parenteral nutrition formulation according to any one of clauses 1 to 20 parenterally (e.g. intravenously) to the neonate.
24. An aqueous neonatal parenteral nutrition formulation according to any one of clauses 1 to 20 for use in reducing the incidence of infections in a neonate.
25. A method of reducing the incidence of infections in a neonate, the method comprising administering an aqueous neonatal parenteral nutrition formulation according to any one of clauses 1 to 20 parenterally (e.g. intravenously) to the neonate.

1. A neonatal parenteral nutrition formulation comprising greater than 14% (w/v) arginine.

2. The neonatal parenteral nutrition formulation according to claim 1, wherein the formulation comprises between 14% (w/v) and 30% (w/v) arginine.

3. The neonatal parenteral nutrition formulation according to claim 1, wherein the formulation comprises between 14% (w/v) and 20% (w/v) arginine.

4. The neonatal parenteral nutrition formulation according to claim 1, wherein the formulation comprises between 14% (w/v) and 16% (w/v) arginine.

5. The neonatal parenteral nutrition formulation according to claim 1, wherein the formulation comprises one or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine or taurine.

6. The neonatal parenteral nutrition formulation according to claim 1, wherein the formulation comprises three or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine or taurine.

7. The neonatal parenteral nutrition formulation according to claim 1, wherein the formulation comprises six or more amino acids selected from isoleucine, leucine, lysine, methionine, phenylalanine, threonine, tryptophan, valine, histidine, alanine, glycine, asparagine, aspartic acid, acetylcysteine, cysteine, glutamic acid, ornithine, proline, serine, tyrosine or taurine.

8. The neonatal parenteral nutrition formulation according to claim 1, wherein the formulation comprises each of the following amino acids in the amounts given:
2.5 to 7.5% (w/v) isoleucine;
7.0 to 11.0% (w/v) leucine;
6.0 to 12.0% (w/v) lysine;
1.0 to 4.0% (w/v) methionine;
2.0 to 5.0% (w/v) phenylalanine;
2.5 to 7.0% (w/v) threonine;
0.5 to 3.5% (w/v) tryptophan;
3.5 to 8.0% (w/v) valine;
1.0 to 5.0% (w/v) histidine;
6.0 to 10.0% (w/v) alanine;
1.0 to 5.0% (w/v) glycine;
0 to 2.0% (w/v) asparagine;
3.5 to 7.0% (w/v) aspartic acid;
0 to 2.0% (w/v) acetylcysteine;
0 to 2.5% (w/v) cysteine;
7.0 to 12.0% (w/v) glutamic acid;
0 to 4.0% (w/v) ornithine;
2.5 to 10.0% (w/v) proline;
2.5 to 6.5% (w/v) serine;
0 to 1.5% (w/v) tyrosine;
0 to 1.5% (w/v) taurine; and
12 to 18% (w/v) arginine.

9. The neonatal parenteral nutrition formulation according to claim 1, wherein the formulation comprises each of the following amino acids in the amounts given:
3.5 to 6.5% (w/v) isoleucine;
9.0 to 10.5% (w/v) leucine;
7.0 to 11.0% (w/v) lysine;
1.0 to 3.0% (w/v) methionine;
3.0 to 4.5% (w/v) phenylalanine;
3.0 to 6.0% (w/v) threonine;
1.0 to 2.5% (w/v) tryptophan;
5.0 to 7.5% (w/v) valine;
2.0 to 4.0% (w/v) histidine;
6.5 to 9.5% (w/v) alanine;
2.5 to 4.0% (w/v) glycine;
0 to 1.0% (w/v) asparagine;
4.5 to 6.0% (w/v) aspartic acid;
0 to 1.0% (w/v) acetylcysteine;
0.5 to 2.0% (w/v) cysteine;
8.5 to 10.5% (w/v) glutamic acid;
0 to 3.5% (w/v) ornithine;
2.5 to 8.0% (w/v) proline;
3.0 to 6.0% (w/v) serine;
1 to 1.0% (w/v) tyrosine;
0 to 1.0% (w/v) taurine; and
14 to 16% (w/v) arginine.

10. The neonatal parenteral nutrition formulation according to claim 1, wherein the formulation comprises one or more electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate.

11. The neonatal parenteral nutrition formulation according to of claim 1, wherein the formulation comprises three or more, or preferably five or more, electrolytes selected from sodium, potassium, magnesium, calcium, selenium, zinc, copper, manganese, phosphate, sulphate, chloride, fluoride, iodide, acetate, citric acid, malate, lactate, glycerophosphate or gluconate.

12. The neonatal parenteral nutrition formulation according to claim 1, wherein the formulation comprises each of the following electrolytes in the amounts given:
0.5 to 100 mmol/L sodium;
0.5 to 80 mmol/L potassium;
0.5 to 10 mmol/L magnesium;
0.5 to 120 mmol/L acetate;
0.5 to 120 mmol/L chloride;
0 to 10 mmol/L calcium;
0 to 40 mmol/L selenium;
0 to 10 mmol/L zinc;
0 to 5 mmol/L copper;
0 to 50 mmol/L manganese;
0 to 20 mmol/L phosphate;
0 to 50 mmol/L fluoride;
0 to 50 mmol/L iodide;
0 to 50 mmol/L glycerophosphate;
0 to 120 mmol/L gluconate; and
0 to 50 mmol/L sulphate.

13. The neonatal parenteral nutrition formulation according to claim 1, wherein the formulation comprises each of the following electrolytes in the amounts given:
0.5 to 100mmol/L sodium;
0.5 to 80 mmol/L potassium;
0.5 to 10 mmol/L magnesium;
0.5 to 120 mmol/L acetate;
0.5 to 120 mmol/L chloride;
0.1 to 10 mmol/L calcium;
0.1 to 40 mmol/L selenium;
0.1 to 10 mmol/L zinc;
0.1 to 5 mmol/L copper;
0.1 to 50 mmol/L manganese;
0.1 to 20 mmol/L phosphate;
0.1 to 50 mmol/L fluoride;
0.1 to 50 mmol/L iodide;
0.1 to 50 mmol/L glycerophosphate;
0.1 to 120 mmol/L gluconate; and
0.1 to 50 mmol/L sulphate.

14. The neonatal parenteral nutrition formulation according to claim 1, wherein the formulation comprises each of the following electrolytes in the amounts given:
60 to 160 mmol/L sodium;
10 to 40 mmol/L potassium;
1.5 to 5 mmol/L magnesium;
30 to 80 mmol/L acetate;
10 to 50 mmol/L chloride;
10 to 20 mmol/L calcium;
20 to 50 mmol/L phosphate;
20 to 50 mmol/L glycerophosphate;
0 to 2 mmol/L selenium;
0 to 1 mmol/L zinc;
0 to 0.5 mmol/L copper;
0 to 1 mmol/L manganese;
0 to 0.5 mmol/L fluoride;
0 to 0.5 mmol/L iodide;
0 to 100 mmol/L gluconate; and
0 to 50 mmol/L sulphate.

15. The neonatal parenteral nutrition formulation according to claim 1, wherein the formulation further comprises one or more carbohydrates.

16. The neonatal parenteral nutrition formulation according to claim 15, wherein the carbohydrate is glucose.

17. The neonatal parenteral nutrition formulation according to claim 16, wherein the formulation comprises between 5 and 20% w/v of glucose.

18. The neonatal parenteral nutrition formulation according to claim 1, characterised in that the formulation comprises less 120 mmol/L of chloride.

19. The neonatal parenteral nutrition formulation according to claim 1, wherein the formulation has any one of the compositions provided under the headings “12%Arginine” and/or “15% Arginine” in Tables 1 and 2.

20. The neonatal parenteral nutrition formulation according to claim 1, wherein the formulation has any one of the compositions provided under the heading “15%Arginine” in Tables 1 and 2.

21. An aqueous neonatal parenteral nutrition formulation according to claim 1, for use in the parenteral nutrition for neonates.

22. The aqueous neonatal parenteral nutrition formulation according claim 21 for use in the treatment of hypoargininaemia, hyperammonaemia, negative nitrogen balance or to prevent weight loss (i.e. to encourage weight gain) in neonates.

23. A method of treating hypoargininaemia, hyperammonaemia, negative nitrogen balance in neonates, or preventing weight loss (i.e. encouraging weight gain) in neonates, the method comprising administering an aqueous neonatal parenteral nutrition formulation according to claim 1 parenterally (e.g. intravenously) to the neonate.

24. The aqueous neonatal parenteral nutrition formulation according to claim 21 for use in reducing the incidence of infections in a neonate.

25. A method of reducing the incidence of infections in a neonate, the method comprising administering an aqueous neonatal parenteral nutrition formulation according to claim 1 parenterally (e.g. intravenously) to the neonate.
